(19) United States
(12) Patent Application Publication
(10) Pub. No.: US 2014/0357946 A1
(43) Pub. Date: Dec. 4, 2014

(54) TISSUE SPREADER FOR ACCESSING PAPILLA, AND RELATED METHODS OF USE

(71) Applicant: Boston Scientific Scimed, Inc., Maple Grove, MN (US)

(72) Inventors: John B. GOLDEN, Norton, MA (US); Paul SMITH, Smithfield, RI (US); Robert B. DeVRIES, Northboro, MA (US); Ryan HARTMAN, Duxbury, MA (US); Michael Killion FORD, Waltham, MA (US); Kenneth BLAIR, Burlington, MA (US); William BERTOLINO, Framingham, MA (US); James WELDON, Newton, MA (US); Laura Elizabeth FIRSTENBERG, Worcester, MA (US); Gary S. KAPPEL, Acton, MA (US); Desiree Dawn BALDERRAMA, Boston, MA (US); Paul MANNION, Shrewsbury, MA (US); James WINDHEUSER, Hopkinton, MA (US); Vanessa NAHON, Boston, MA (US); Kenny Joe KING, Somerville, MA (US); Heather A. SIMANI, Dedham, MA (US); Ritesh DAHYA, Albuquerque, NM (US); Kerry L. GRANT, Northbridge, MA (US)

(73) Assignee: Boston Scientific Scimed, Inc., Maple Grove, MN (US)

(21) Appl. No.: 14/294,888

(22) Filed: Jun. 3, 2014

Related U.S. Application Data
(60) Provisional application No. 61/831,009, filed on Jun. 4, 2013.

(51) Int. Cl. A61B 17/02; A61B 1/018

(52) U.S. Cl. CPC A61B 17/0206; A61B 1/018
USPC 600/104; 600/214

(57) ABSTRACT

The medical device may include an elongate member having a proximal end and a distal end, and a plurality of legs coupled to the distal end of the elongate member. The medical device may also include a moveable element distal to the distal end of the elongate member, disposed around the plurality of legs. The moveable element may be reciprocally moveable relative to the elongate member and plurality of legs between a first and second position. The plurality of legs may be in a collapsed configuration when the moveable element is in the first position, and may be in an expanded configuration when the moveable element is in the second position.

### CROSS-REFERENCE TO RELATED APPLICATIONS

This application claims the benefit of priority of U.S. Provisional Application No. 61/831,009, filed Jun. 4, 2013, the entirety of which is incorporated by reference herein.

### TECHNICAL FIELD

Various embodiments of the present disclosure relate generally to medical devices and related systems and methods. More specifically, the present disclosure relates to devices, systems, and methods for accessing the pancreaticobiliary system, e.g., to examine, diagnose, and/or treat a condition of the pancreatic duct or the bile duct.

### BACKGROUND

Access to the pancreaticobiliary system is required to diagnose and/or treat a variety of conditions, including tumors, gallstones, infection, sclerosis, and pseudocysts. One method of gaining access is via endoscopic retrograde cholangiopancreatography (ERCP), in which a side-viewing endoscope is passed down the esophagus, through the stomach, and into the duodenum where the duodenal papilla leading into the pancreatic and bile ducts may be visualized. In ERCP, tools such as sphincterotomes are passed through the working channel of the scope to gain access to the papilla, e.g., to investigate potential obstruction or inflammation of the pancreatic or bile ducts. Fluoroscopic contrast may be injected into either duct and X-ray images taken to determine the presence and location of strictures or stones.

Cannulation of either the bile duct or the pancreatic duct is a significant challenge in ERCP procedures. Factors that may complicate insertion into the papilla include sphincter orientation, floppy intraductal segments, biliary/pancreatic take-off levels, and the presence of stones or strictures. Difficult cannulations carry a high risk of perforation or other damage to tissue. For example, one technique physicians use to cannulate the papilla is to identify a bile trail, e.g., by pushing against the ampulla or applying suction to encourage bile from the duct. Prolonged probing, however, may lead to inflammation of the papilla and adverse effects for the patient.

Complications also may arise when the duct accessed first is not the duct desired for the procedure. When biliary access is desired, for example, a physician first may gain access to the pancreatic duct, e.g., via a guide wire. The physician then would have to remove the wire and attempt cannulation again. The pancreatic duct may be entered unintentionally several more times before access to the bile duct is finally achieved. These multiple pancreatic injections can irritate the tissue of the pancreatic duct and cause post-ERCP complications such as pancreatitis.

### SUMMARY

The present disclosure includes devices, systems, and methods for cannulating the pancreatic and biliary ducts such as during an ERCP procedure.

In accordance with an embodiment, a medical device may include an elongate member having a proximal end and a distal end, and a plurality of legs coupled to the distal end of the elongate member. The medical device may also include a moveable element distal to the distal end of the elongate member, disposed around the plurality of legs. The moveable element may be reciprocally moveable relative to the elongate member and plurality of legs between a first and second position. The plurality of legs may be in a collapsed configuration when the moveable element is in the first position, and may be in an expanded configuration when the moveable element is in the second position.

Various embodiments of the disclosure may include one or more of the following aspects: wherein the second position is proximal to the first position; wherein the plurality of legs extend radially outward to move from the collapsed configuration to the expanded configuration; wherein in the collapsed configuration, free distal ends of the plurality of legs are converged to penetrate a papilla; wherein free distal ends of each of the plurality of legs includes a plurality of protrusions configured to engage bodily tissue atraumatically; wherein the plurality of legs are substantially parallel to a longitudinal axis of the medical device while in the collapsed configuration; wherein each of the plurality of legs includes a branch member at a distal end, wherein the branch member is angled toward the longitudinal axis; wherein each of the plurality of legs has a wavy shape in the expanded configuration, and a pad coupled to each branch member; wherein the plurality of legs form a cylindrical, distal tip in the collapsed configuration, wherein the moveable element is on a plurality of moveable elements, and at least one moveable element is coupled to each of the plurality of legs; further including a coating coupled to each of the plurality of legs, the coating including a plurality of protrusions; and wherein each of the plurality of protrusions has a diameter from about 1 μm to about 100 μm.

In accordance with an embodiment of the present disclosure, a method of using a medical device may include inserting an elongate member having a proximal end and a distal end into a body passage, and directing a plurality of legs coupled to the distal end of the elongate member toward a papilla while the plurality of legs are in a collapsed configuration. The method may also include moving a moveable element distal to the distal end of the elongate member from a first position to a second position to cause the plurality of legs to move from the collapsed configuration to an expanded configuration.

Various embodiments of the present disclosure may include one or more of the following aspects: inserting a distalmost end of each of the plurality of legs into the papilla while the plurality of legs are in the collapsed configuration; moving the moveable element from the first position to the second position while the distalmost end of each of the plurality of legs is within the papilla; and wherein the plurality of legs grasp and stabilize bodily tissue surrounding the papilla.

In accordance with an embodiment of the present disclosure, a method for expanding a papilla may include inserting a tube into a body passage, the tube having a proximal end, a distal end, and a lumen extending between the proximal and distal ends, and directing an elongate member disposed within the lumen through a distal end of the tube and into the papilla. The method may also include directing a plurality of legs disposed at a distal end of the elongate member between a collapsed configuration and an expanded configuration. The plurality of legs may be biased toward a longitudinal axis of the elongate member in the collapsed configuration, and may expand radially outward from the longitudinal axis in the expanded configuration.

Various embodiments of the present disclosure may include inserting the tube and the elongate member through an endoscope.

In accordance with an embodiment of the present disclosure, a method of accessing a papilla may include inserting an elongate member having a proximal end and a distal end into the papilla, wherein the elongate member includes a coating disposed at the distal end, the coating having a plurality of protrusions having a diameter from about 1 μm to about 100 μm.

Various embodiments of the present disclosure may include one or more of the following aspects: wherein the coating is configured to be lubricous against bodily tissue; and wherein the coating is configured to be tacky against bodily tissue; and then removing the elongate member from the papilla to expand a diameter of a hepaticopancreatic ampulla connected to the papilla.

In accordance with an embodiment of the present disclosure, a method of accessing a papilla may include directing a movable member from the distal end of an elongate member toward the papilla. The method may also include actuating a distal end of the movable member to direct a hoop from a collapsed configuration to an expanded configuration, and positioning the hoop around the papilla while the hoop is in the expanded configuration.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 shows anatomical features of the pancreaticobiliary system.

FIGS. 2A-2B are partial side views of a medical device for spreading tissue in accordance with an embodiment of the present disclosure.

FIGS. 3A-3B illustrate in vivo perspective views of the medical device of FIGS. 2A-2B in accordance with an embodiment of the present disclosure.

FIGS. 4A-4B are partial side views of a medical device for spreading tissue in accordance with an embodiment of the present disclosure.

FIGS. 5A-5B illustrate in vivo perspective views of the medical device of FIGS. 4A-4B in accordance with an embodiment of the present disclosure.

FIGS. 6A-6B are partial side views of a medical device for stabilizing tissue in accordance with an embodiment of the present disclosure.

FIGS. 7A-7B illustrate in vivo perspective views of the medical device of FIGS. 6A-6B in accordance with an embodiment of the present disclosure.

FIGS. 8A-8B are partial side views of a medical device for spreading and/or stabilizing tissue in accordance with an embodiment of the present disclosure.

FIGS. 9A-9C illustrate in vivo perspective views of the medical device of FIGS. 8A-8B in accordance with an embodiment of the present disclosure.

FIGS. 10A-10C illustrate perspective views of a medical device for spreading tissue in accordance with an embodiment of the present disclosure.

FIGS. 11A-11B illustrate in vivo perspective views of the medical device of FIGS. 10A-10C in accordance with an embodiment of the present disclosure.

FIGS. 12A-12B illustrate in vivo perspective views of a medical device in accordance with an embodiment of the present disclosure.

FIG. 13 is a partial side view of a coating in accordance with an embodiment of the present disclosure.

FIG. 14 is a partial side view of a medical device having the coating of FIG. 13 in accordance with an embodiment of the present disclosure.

FIG. 15A is a top plan view of a medical device in accordance with an embodiment of the present disclosure.

FIG. 15B is a partial side view of the medical device of FIG. 15A.

FIG. 15C is a top plan view of a medical device in accordance with an embodiment of the present disclosure.

FIG. 15D is a partial side view of the medical device of FIG. 15C.

FIGS. 16A-16C are perspective views of medical devices in accordance with various embodiments of the present disclosure.

FIG. 17 illustrates an in vivo perspective view of the medical device of FIG. 16A in accordance with an embodiment of the present disclosure.

FIG. 18 is a partial side view and an end view of a medical device for stabilizing tissue in accordance with an embodiment of the present disclosure.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts.

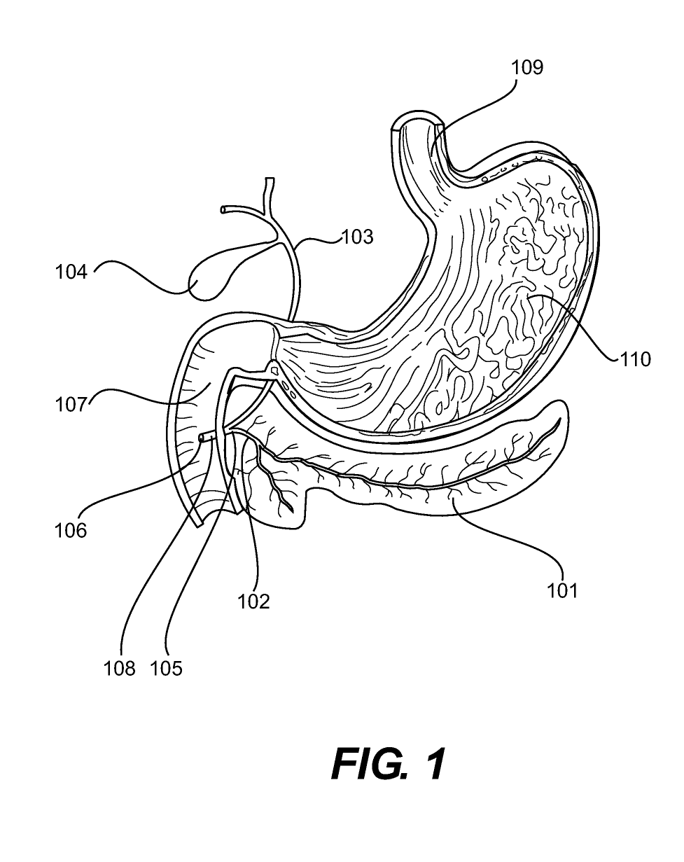

The pancreaticobiliary system, illustrated in FIG. 1, includes the pancreas 101, the pancreatic duct 102, the common bile or biliary duct 103, and the gallbladder 104. The pancreatic and biliary ducts join at the hepatopancreatic ampulla 105 (also known as the ampulla of Vader), which lies just behind the papilla 106. The papilla is a small opening that leads into the duodenum 107 to allow for the release of pancreatic juice and bile into the duodenum to aid in digestion. Smooth muscle of the hepatopancreatic sphincter 108 (also known as the sphincter of Oddi) regulates flow of pancreatic juice and bile into the duodenum.

ERCP procedures often use a guidewire and catheter through the working channel of an endoscope to access an area of interest within the pancreatic duct or bile duct, e.g., for examination, diagnosis, and/or treatment. A guidewire may pass through the working channel of the endoscope and allow for exchange of a catheter, such as a sphincterotome, over the wire. Guidewires are available in a variety of diameters, e.g., ranging from about 0.018″ to about 0.035″ outer diameter, and typically include a solid metallic core with an applied coating. The coating may have markings for visual indicators and may provide a lubricious surface for catheters passed over the wire. The tip of the guidewire may be tapered and constructed of a softer material to promote cannulation of the papilla and minimize trauma to the patient. Guidewires generally are provided at sufficient length to allow passage through the working channel of the endoscope and allow the exchange of catheters over the wire.

Sphincterotomes are catheters that may be introduced via the working channel of an endoscope to cannulate the papilla, and may include a tapered tip, e.g., ranging from about 3.5 Fr to about 5.5 Fr, to ease cannulation of the papilla. A sphincterotome may have one or more lumens for receiving a guidewire or injecting contrast, and generally includes an electrosurgical cutting wire at the tip to provide transmission of high frequency electrical current to incise the sphincter.

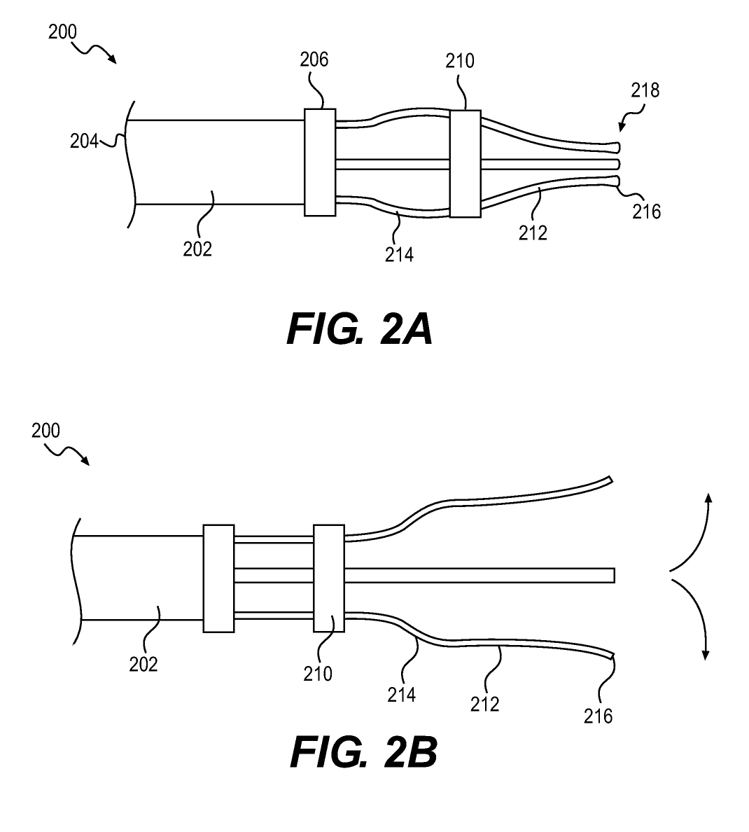

FIGS. 2A-2B show different partial side views of a medical device for spreading tissue in accordance with an embodiment of the present disclosure.

As shown in FIG. 2A, a medical device 200 may include an elongate shaft 202, having a proximal end 204 and a distal end 206. The elongate shaft 202 may be inserted into a lumen of a patient's body to access a target site whereupon a medical or diagnostic procedure is performed. The proximal end 204 may be connected to an actuating mechanism (not shown) for operating the medical device 200. The actuating mechanism may be any appropriate mechanism for controlling and operating the components of medical device 200 to enable spreading of tissue using the medical device. In one embodiment, the actuating mechanism may be a handle, configured to be driven either manually, or through a pneumatic, hydraulic, or other electromechanical mechanism.

A moveable element 210 may be disposed around a plurality of legs 212. Moveable element 210 may be a ring, sheath, or another suitable shape configured to be moveable (e.g., slideable and/or axially translatable) with respect to the plurality of legs 212. Moveable element 210 may be coupled to an actuation member (not shown) that extends partially through the elongate shaft 202, and further, may extend outward from the distal end 206 of the elongate shaft 202. Moveable element 210 may further be disposed around the actuation member, and may be coupled to the actuation member by a plurality of links, such as, e.g., rods, wires, threads, or other suitable links. It should be noted however that other suitable actuation members and coupling mechanisms are also contemplated. Moveable member 210 may reciprocate between a first position and a second position.

Each leg 212 may have a proximal end 214 and a free distal end 216. The plurality of legs 212 may extend distally from distal end 206 of medical device 200. That is, proximal end 214 of each leg 212 may be coupled to distal end 206, or alternatively may be disposed partially within shaft 202, of medical device 200. Any suitable mechanism may be used to secure proximal ends 214 to distal end 206, including, e.g., sealing or adhesively bonding. The plurality of legs 212 may be disposed around a circumference of distal end 206. Any number of suitable legs 212 may be used, and legs 212 may be disposed approximately equidistant from each other, if desired. In one exemplary embodiment, four legs 212 are utilized. Legs 212 may be generally S-shaped, or wavy, and thus may bow outward from a longitudinal axis and then bow inward toward the longitudinal axis when extending from distal end 206 in the distal direction. The free distal ends 216 of the plurality of legs 212 may be unconnected to each other or to any other part of medical device 200.

Referring to FIG. 2A, the plurality of legs 212 are depicted in a first, retracted (or, collapsed) configuration such that the distal ends 216 of the legs 212 may converge generally at a point 218 so that legs 212 are configured to be inserted into a small opening of a body. While the plurality of legs 212 are in the first, retracted configuration, moveable element 210 may be disposed between proximal and distal ends 214, 216 of legs 212 in the first position.

Referring to FIG. 2B, the plurality of legs 212 are depicted in a second, extended (or, expanded) configuration. In this embodiment, the plurality of legs 212 may extend radially outward from a longitudinal axis of the medical device 200. While the plurality of legs 212 are in the second, extended configuration, moveable element 210 may be disposed in the second position closer to proximal end 214 and further from distal end 216 than the first position.

Moveable element 210 may be configured to reciprocally move between the first and second positions. That is, a user may direct moveable element 210 via, e.g., the actuating mechanism, from the first position to the second position in order to cause the plurality of legs 212 to move from the first, retracted configuration to the second, extended configuration. Similarly, a user may also direct moveable element 210 from the second position to the first position in order to cause the plurality of legs 212 to move from the second, extended configuration to the first, retracted configuration.

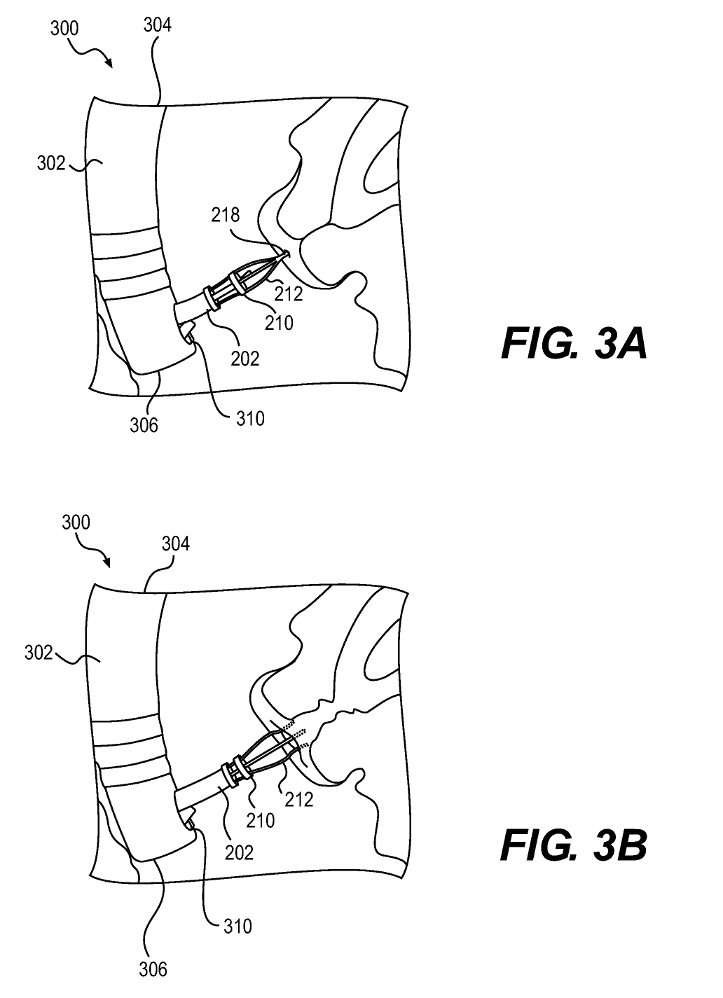

FIGS. 3A-3B illustrate in vivo perspective views of medical device 200. Specifically referring to FIG. 3A, medical device 200 may be used with an endoscopic device 300. Endoscopic device 300 may be used for dissecting and/or resecting polyps, lesions, or other unwanted tissue from the interior bodily walls or other anatomical structure of a patient, or for any other medical procedure. Endoscopic device 300 may include an elongate member 302 having a proximal end 304, a distal end 306, and a lumen (not shown) extending between proximal and distal ends 304, 306. Proximal end 304 may be coupled to an appropriate handle (not shown), while distal end 306 may include one or more openings 310 in communication with the lumen. In the embodiment shown, opening 310 is shown as disposed in a side wall at distal end 306 of elongate member 302. However, it should be noted that additional or alternative opening(s) 310 may be disposed at other locations, such as, but not limited to a distal end face of endoscopic device 300 or any other suitable location.

Endoscopic device 300 may be an endoscope or any other suitable introduction device or sheath adapted to be advanced into a body lumen. For example, endoscopic device 300 may include a guide tube, an endoscope, a guide sheath, a flexible member, and/or a catheter. In the illustrated embodiment, the lumen of elongate member 302 may include one or more channels (not shown), through which the operator may introduce one or more medical devices to extend out of distal end 306 of elongate member 302. For example, during a tissue dissection or resection procedure, the operator may introduce a suction device into one channel and a cutting device, such as, for example, a snare loop, into another channel. Additionally, the operator may insert a light source, a camera, an injector, or a morcellator within the one or more channels. Because different implements may need to be inserted into the elongate member 302, the dimensions of its channels may vary. Some channels may have a larger diameter, while others may have a smaller diameter. Further, some channels may include permanently fixed devices, such as light sources or imaging devices extending to a distal face of elongate member 302, while other channels may allow temporary insertion and removal of medical devices, as the operator desires. Elongate member 302 also may be any known endoscopic device used, for example, for colonoscopy, resectoscopy, cholangioscopy, tissue dissection, or mucosal resection.

Moreover, elongate member 302 may be coated with lubricious materials and antibacterial agents to ease insertion into tight cavities and prevent infections, respectively. Further, portions of the elongate member 302 may include radiopaque materials to visualize the position of elongate member 302 within a patient's body.

Medical device 200 may extend through the lumen of endoscopic device 300 and out of endoscopic device 300 via opening 310. Because opening 310 may be located on a side wall of endoscopic device 300, medical device 200 may extend substantially perpendicular to the longitudinal axis of endoscopic device 300. Endoscopic device 300 may be inserted through a natural opening of a patient, such as, for example, the mouth or anus, to reach a desired internal anatomy, or may be inserted through a percutaneous incision. Specifically referring to FIG. 3A, endoscopic device 300 may be placed in proximity to the papilla such that opening 310 may be generally aligned with the papilla. Medical device 200 may then be directed out of opening 310 such that point 218 penetrates the papilla. When point 218 first penetrates the papilla, moveable element 210 may be in the first position, while legs 212 may be in the first, retracted configuration.

Referring to FIG. 3B, once point 218 has penetrated the papilla, a user may direct moveable element 210 to the second position, causing legs 212 move to the second, extended configuration, where legs 212 expand radially outward about the longitudinal axis of medical device 200 against the interior walls of the hepatopancreatic ampulla, and widen the papilla to facilitate ERCP. A guidewire (not shown) may be passed through medical device 200 to gain access to the common bile or biliary duct and/or the pancreatic duct.

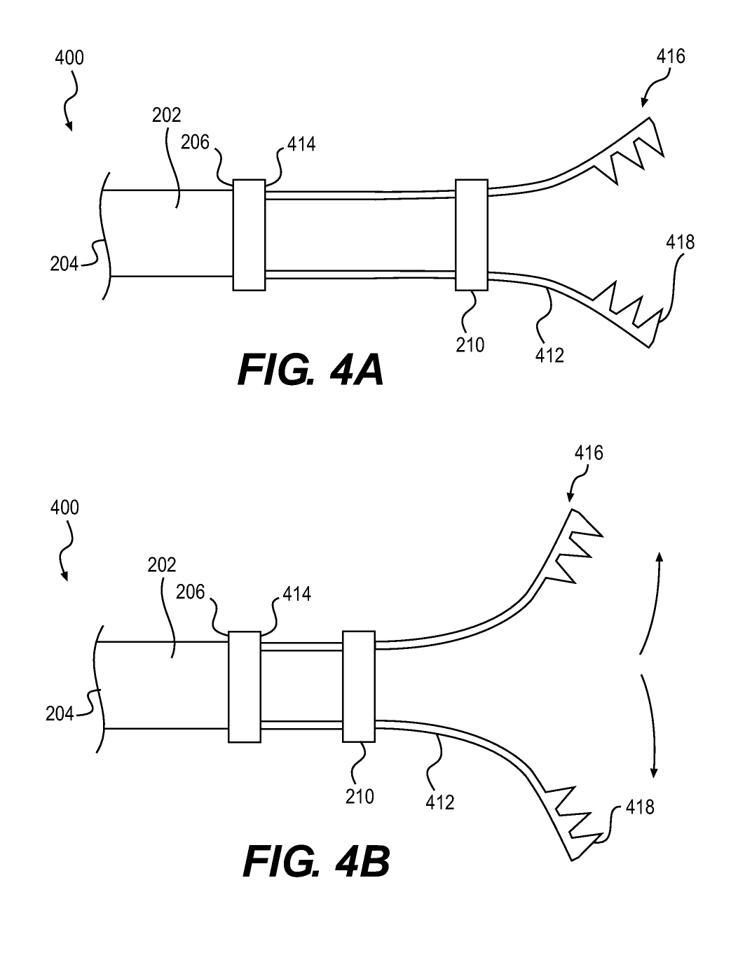

FIGS. 4A-4B show different partial side views of a medical device for spreading tissue in accordance with an alternative embodiment of the present disclosure.

As shown in FIG. 4A, a medical device 400 may include an elongate shaft 202 having a proximal end 204 and a distal end 206, an actuating mechanism (not shown), and a moveable element 210 disposed around a plurality of legs 412. Each leg 412 may have a proximal end 414 and a free distal end 416.

The plurality of legs 412 may extend distally from distal end 206 of medical device 400. That is, proximal end 414 of each leg 412 may be coupled to distal end 206, or alternatively may be partially disposed within shaft 202, of medical device 400. Any suitable mechanism may be used to secure proximal ends 214 to distal end 206, including, e.g., sealing or adhesively bonding. The plurality of legs 412 may be disposed around a circumference of distal end 206. Any number of suitable legs 412 may be used, and legs 412 may be disposed approximately equidistant from each other, if desired. In one exemplary embodiment, two legs 412 are utilized. Legs 412 may be generally straight at a proximal portion, and may have a pre-shaped bend so that each leg 412 arcs away from the a longitudinal axis of medical device 400 toward distal end 416. Each leg 412 may also include a plurality of protrusions 418 configured to attach to or grasp bodily tissue in an atraumatic fashion that does not irritate the papilla. That is, protrusions 418 are configured to attach to bodily tissue yet may not cause any or significant damage to the bodily tissue. Protrusions 418 may include barbs, ridges, prongs, or any other suitable protrusion configured to attach to the bodily tissue in an atraumatic fashion. While in the embodiments shown in FIGS. 4A and 4B, protrusions 418 are inwardly-facing, in an alternative embodiment, protrusions 418 may instead be outwardly-facing in order to apply a greater outward force, improving access to the papilla.

Referring to FIG. 4A, the plurality of legs 412 are depicted in a first, retracted configuration. The first, retracted configuration additionally could have legs 412 in a completely straight configuration. While the plurality of legs 412 are in the first, retracted configuration, moveable element 210 may be disposed between proximal and distal ends 414, 416 of legs 412 in the first position.

Referring to FIG. 4B, the plurality of legs 412 are depicted in a second, extended configuration. In the second, extended configuration, the plurality of legs 412 may extend radially outward from the longitudinal axis of the medical device 400. While the plurality of legs 412 are in this second, extended position, moveable element 210 may be located at a second position closer to proximal end 414 and further from distal end 416, as compared to the first position.

In one embodiment, moveable element 210 may be configured to reciprocally move between the first and second positions. That is, a user may direct moveable element 210 via, e.g., the actuating mechanism, from the first position to the second position, causing the plurality of legs 412 to move from the first, retracted configuration to the second, extended configuration. Similarly, a user may also direct moveable element 210 from the second position to the first position, causing the plurality of legs 412 to move from the second, extended configuration to the first, retracted configuration.

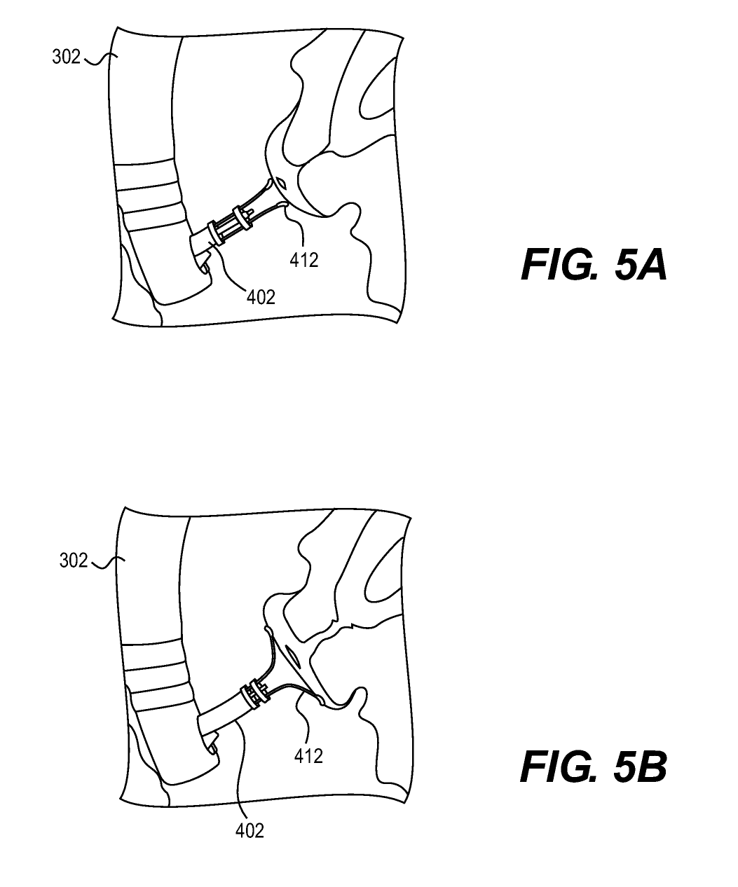

FIGS. 5A-5B illustrate various in vivo perspective views of medical device 400. Specifically referring to FIG. 5A, medical device 400 may be used with endoscopic device 300 described above with reference to FIGS. 3A and 3B. Medical device 400 may extend through the lumen of endoscopic device 300 and out of endoscopic device 300 via opening 310. Because opening 310 may be located on a side wall of endoscopic device 300, medical device 400 may extend substantially perpendicular to the longitudinal axis of endoscopic device 300. Medical device 400 may be directed out of opening 310 toward the papilla such that protrusions 418 may engage the bodily tissue surrounding the papilla. While protrusions 418 first grasp bodily tissue surrounding the papilla, it should be noted that moveable element 210 may be in the first position, while legs 412 may be in the first, retracted configuration.

Referring to FIG. 5B, once protrusions 418 have engaged the bodily tissue surrounding the papilla, a user may direct moveable element 210 to the second position, such that legs 412 expand radially outward about the longitudinal axis of medical device 400. As legs 412 expand outward, the bodily tissue surrounding the papilla is similarly expanded outward, widening the papilla. A guidewire (not shown) may be passed through medical device 400 to gain access to the hepatopancreatic ampulla. The opening and restraining of the tissue can help hold the papilla in position to allow the guidewire to gain access into the common bile or biliary duct and/or the pancreatic duct.

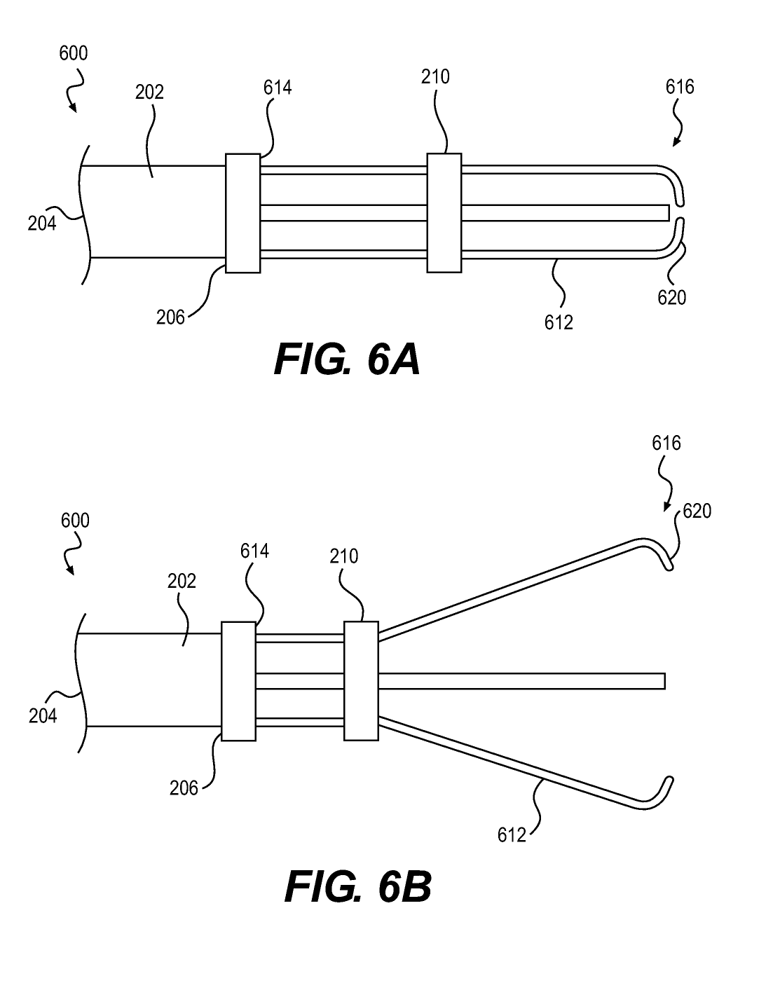

FIGS. 6A-6B show different partial side views of a medical device for stabilizing tissue in accordance with an alternative embodiment of the present disclosure.

As shown in FIG. 6A, a medical device 600 may include an elongate shaft 202 having a proximal end 204 and a distal end 206, an actuating mechanism (not shown), and a moveable element 210 disposed around a plurality of legs 612. Each leg 612 may have a proximal end 614 and a distal end 616.

The plurality of legs 612 may extend distally from distal end 206 of medical device 600. That is, proximal end 614 of each leg 612 may be coupled to distal end 206, or alternatively may be partially disposed within shaft 202, of medical device 600. Any suitable mechanism may be used to secure proximal ends 614 to distal end 206, including, e.g., sealing or adhesively bonding. The plurality of legs 612 may be disposed around a circumference of distal end 206. Any number of suitable legs 612 may be used, and legs 612 may be disposed approximately equidistant from each other, if desired. In one exemplary embodiment, four legs 612 are utilized. Legs 612 may be generally straight at a proximal portion, and include a branch member 620 at distal end 616 of legs 612. Branch member 620 may be substantially perpendicular, or angled, with respect to a length of legs 612. Legs 612 may have a pre-shaped bend along an intermediate portion so that each leg 612 arcs away from the a longitudinal axis of medical device 600 toward distal end 616 at the intermediate portions. While in the embodiments shown in FIGS. 6A and 6B, branch members 620 are inwardly-facing, in an alternative embodiment, branch members 620 may instead be outwardly-facing.

Referring to FIG. 6A, the plurality of legs 612 are depicted in a first, retracted configuration such that lengths of legs 612 may be substantially parallel to the longitudinal axis of medical device 600. While the plurality of legs 612 are in the first, retracted configuration, moveable element 210 may be disposed between proximal and distal ends 614, 616 of legs 612 in a first position.

Referring to FIG. 6B, the plurality of legs 612 are depicted in a second, extended configuration. In the second, extended configuration, the plurality of legs 612 may extend radially outward from the longitudinal axis of the medical device 600. While the plurality of legs 612 are in the second, extended configuration, moveable element 210 may be located at a second position closer to proximal end 614 and further from distal end 616 than the first position.

In one embodiment, moveable element 210 may be configured to reciprocally move between the first and second positions. That is, a user may direct moveable element 210 via, e.g., the actuating mechanism, from the first position to the second position, causing the plurality of legs 612 to move from the first, retracted configuration to the second, extended configuration. Similarly, a user may also direct moveable element 210 from the second position to the first position, causing the plurality of legs 612 to move from the second, extended configuration to the first, retracted configuration.

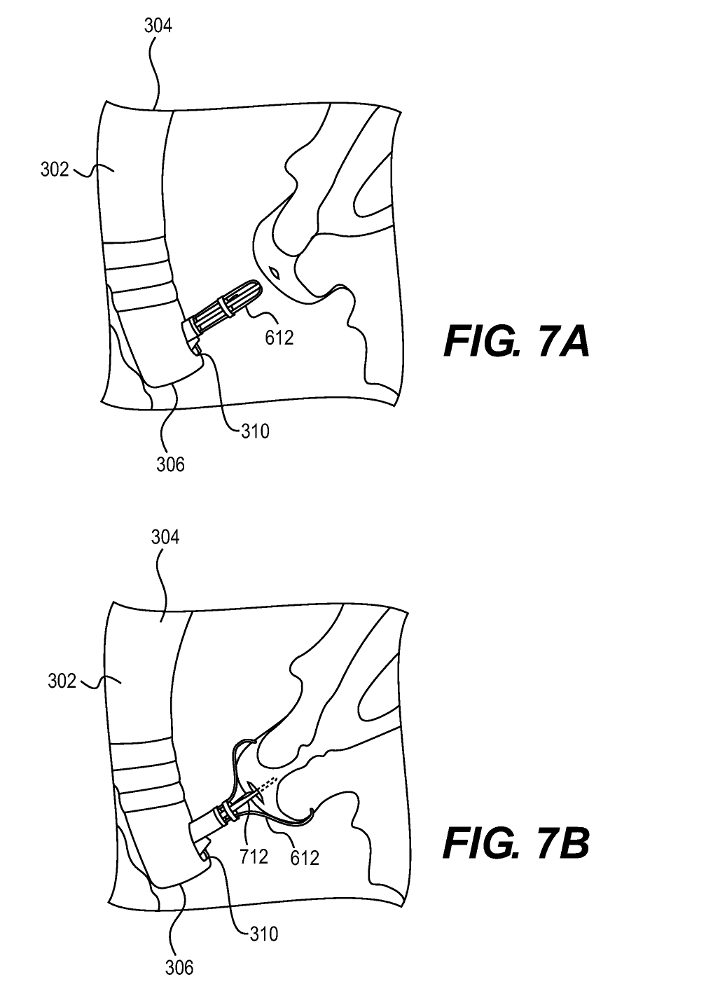

FIGS. 7A-7B illustrate in vivo perspective views of a medical device 600. Specifically referring to FIG. 7A, medical device 600 may be used with endoscopic device 300 described above with reference to FIGS. 3A and 3B. Medical device 600 may extend through the lumen of endoscopic device 300 and out of endoscopic device 300 via opening 310. Because opening 310 may be located on a side wall of endoscopic device 300, medical device 600 may extend substantially perpendicular to the longitudinal axis of endoscopic device 300. Medical device 600 may be directed out of opening 310 toward the papilla while legs 612 are in the retracted position.

Referring to FIG. 7B, a user may direct moveable element 210 to the second position, such that legs 612 expand radially outward about the longitudinal axis of medical device 600. As legs 612 expand outward, branch members 620 may surround and stabilize an outer circumference of bodily tissue surrounding the papilla. For example, branch members 620 may surround and isolate the papilla, preventing movement of the papilla by other bodily forces. Branch members 620 may include a tip that assists in grasping the bodily tissue surrounding the papilla including, but not limited to roughened surfaces, hooks, and barbs. While the bodily tissue is stabilized, a user may access the papilla via a tool 712 disposed in a lumen of medical device 600. Alternatively, tool 712 may be disposed within a lumen of endoscopic device 300, or at another suitable location. A guidewire (not shown) may be passed through medical device 600 to gain access to the hepaticopancreatic ampulla.

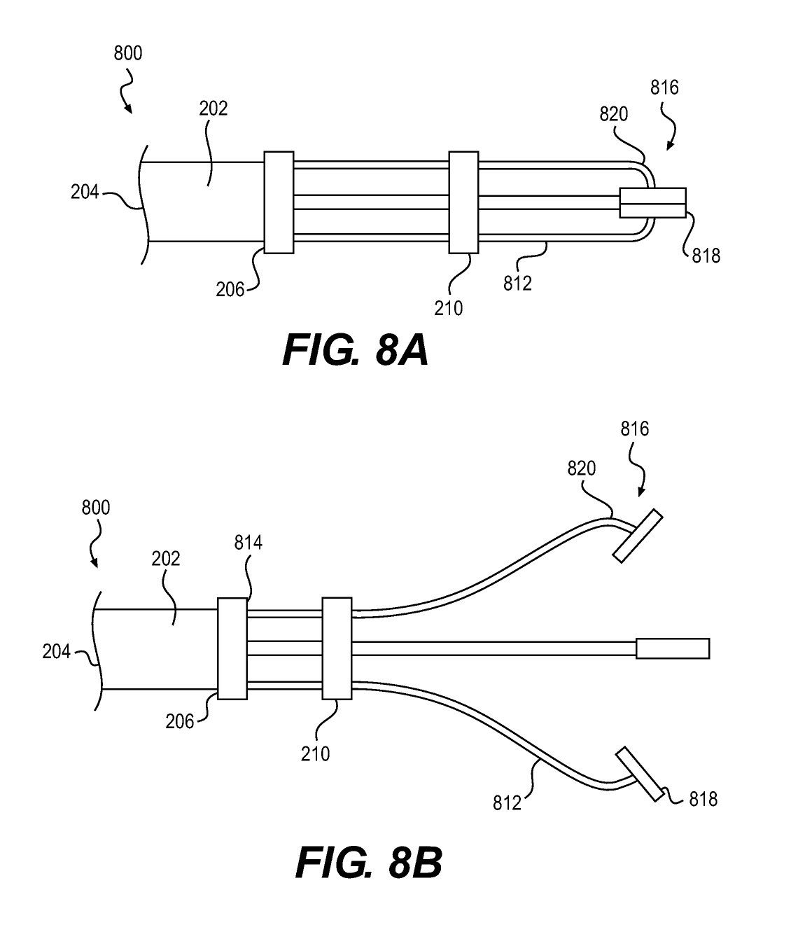

FIGS. 8A-8B show different partial side views of a medical device for spreading and/or stabilizing tissue in accordance with an alternative embodiment of the present disclosure.

As shown in FIG. 8A, a medical device 800 may include an elongate shaft 202 having a proximal end 204 and a distal end 206, an actuating mechanism (not shown), and a moveable element 210 disposed around a plurality of legs 812. Each leg 812 may have a proximal end 814 and a distal end 816.

The plurality of legs 812 may extend distally from distal end 206 of medical device 800. That is, proximal end 814 of each leg 812 may be coupled to distal end 206, or alternatively may be disposed partially within shaft 202, of medical device 800. Any suitable mechanism may be used to secure proximal ends 814 to distal end 206, including, e.g., sealing or adhesively bonding. The plurality of legs 812 may be disposed around a circumference of distal end 206. Any number of suitable legs 812 may be used, and legs 812 may be disposed approximately equidistant from each other, if desired. In one exemplary embodiment, four legs 812 are utilized. Legs 812 may be generally straight at a proximal portion, and include a branch member 820 at distal end 816. Branch member 820 may be substantially perpendicular, or angled, with respect to a length of legs 812. A pad 818 may be attached to an end of branch member 820. Pad 818 may include feet and/or other materials configured to atraumatically anchor bodily tissues surrounding the papilla. Pad 818 also may be generally atraumatic to bodily tissue, but with a surface that can grasp (e.g., is roughened, sticky, tacky, or contains hooks, barbs, or the like). Pads 818 may be generally square, rectangular, round, or have another suitable shape.

Referring to FIG. 8A, the plurality of legs 812 are depicted in a first, retracted configuration such that lengths of legs 812 are substantially parallel to the longitudinal axis of medical device 800. While the plurality of legs 812 are in the first, retracted configuration, moveable element 210 may be disposed between proximal and distal ends 814, 816 of legs 812 at a first position.

Referring to FIG. 8B, the plurality of legs 812 are depicted in a second, extended configuration. In the second, extended configuration, the plurality of legs 812 may extend radially outward from the longitudinal axis of the medical device 800. While the plurality of legs 812 are in the second, extended configuration, moveable element 210 may be located at a second position closer to proximal end 814 and further from distal end 816 than the first position.

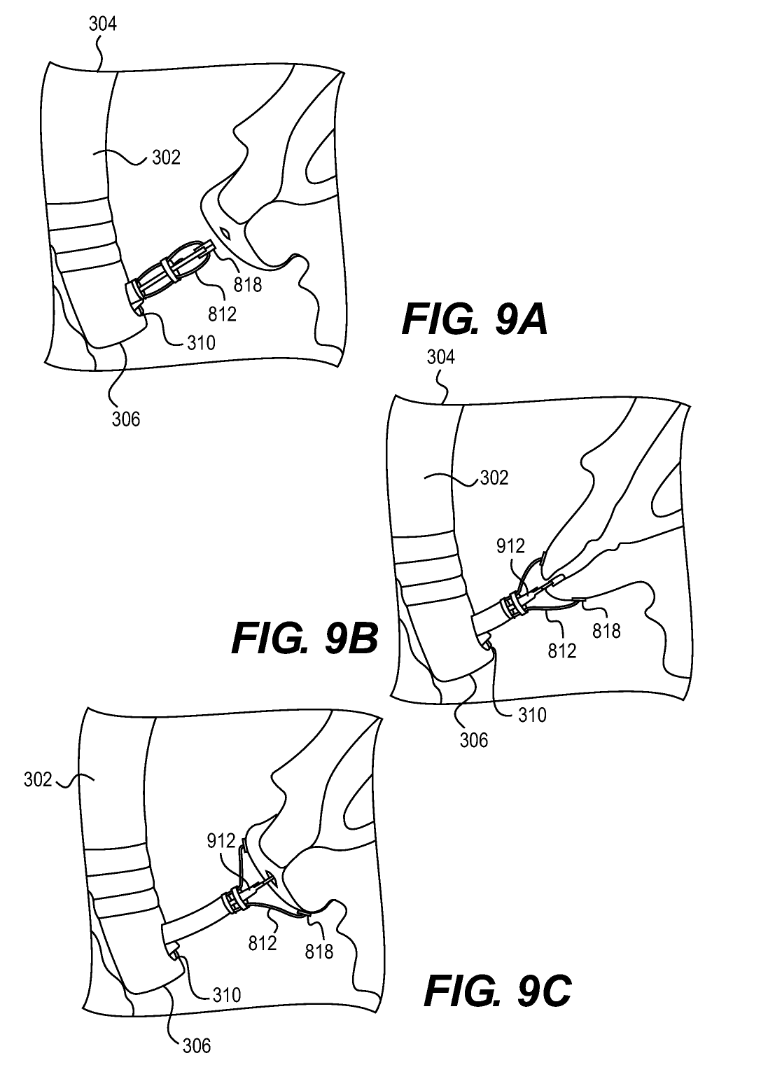

FIGS. 9A-9C illustrate in vivo perspective views of medical device 800. Specifically referring to FIG. 9A, medical device 800 may be used with endoscopic device 300 described above with reference to FIGS. 3A and 3B. Medical device 800 may extend through the lumen of endoscopic device 300 and out of endoscopic device 300 via opening 310. Because opening 310 may be located on a side wall of endoscopic device 300, medical device 800 may extend substantially perpendicular to the longitudinal axis of endoscopic device 300. Medical device 800 may be directed out of opening 310 toward the papilla while legs 812 are in the first, retracted configuration.

Referring to FIG. 9B, a user may direct moveable element 210 to the second, extended position, such that legs 812 expand radially outward about the longitudinal axis of medical device 800. As legs 812 expand outward, branch members 820 and pads 818 may surround and stabilize an outer circumference of bodily tissue surrounding the papilla. For example, branch members 820 may surround and isolate the papilla, preventing movement of the papilla by other bodily forces. Branch members 820 may include a tip that assists in grasping the bodily tissue surrounding the papilla including, but not limited to roughened surfaces, hooks, and barbs. While the bodily tissue is stabilized, a user may access the papilla via a tool 912 disposed in a lumen of medical device 800. Alternatively, tool 912 may be disposed within a lumen of endoscopic device 300, or at another suitable location.

Referring to FIG. 9C, a user may direct moveable element 210 to a third position that is closer to proximal end 814 and further from distal end 816 than both the first and second positions. When moveable element 210 is in the third position, legs 812 may be disposed in a third, extended configuration where legs 812 are further extended radially away from the longitudinal axis of medical device 800. In this embodiment, a user may direct pads 818 to contact the bodily tissue surrounding the papilla either before directing moveable element 210 from the first position to the third position, or while moveable element 210 is in between the first and third positions. After pads 818 are in contact with the bodily tissue surrounding the papilla, the user may direct moveable element 210 to the third position, causing legs 812 to expand outward. As legs 812 expand outward, due to the contact of legs 812 with the bodily tissue around the papilla, the bodily tissue surrounding the papilla is similarly expanded outward, widening the papilla. A guidewire (not shown) may be passed through medical device 800 to gain access to the pancreatic duct.

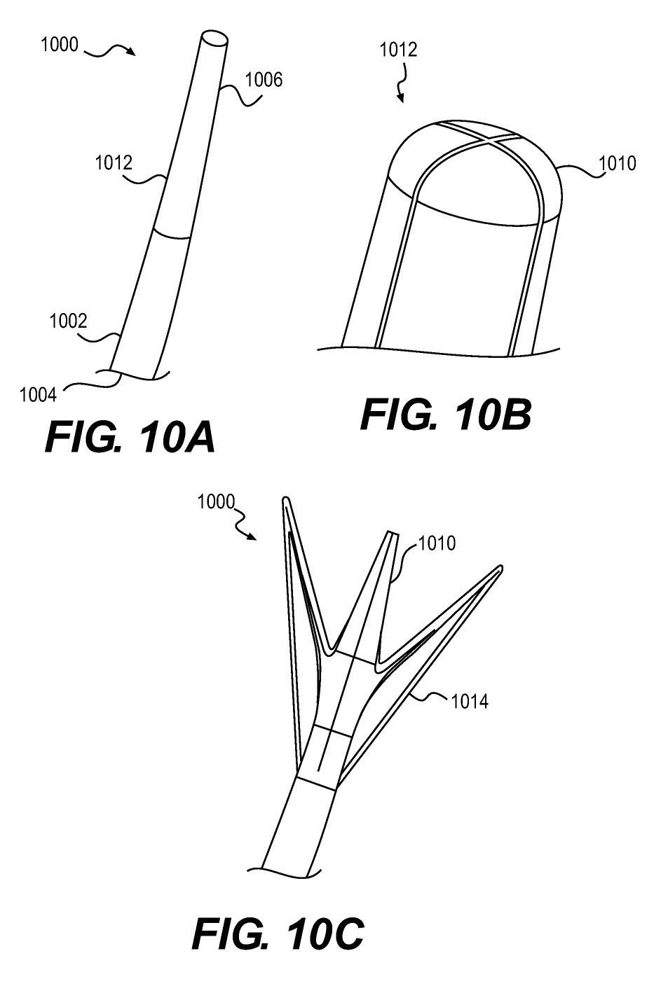

FIGS. 10A-10C show different partial side views of a medical device for spreading tissue in accordance with an alternative embodiment of the present disclosure.

As shown in FIGS. 10A-10C, a medical device 1000 may include an elongate shaft 1002 having a proximal end 1004 and a distal end 1006, an actuating mechanism (not shown), and a plurality of legs 1010 that together form a distal tip 1012. An actuation member 1014 may be coupled to each leg 1010, and may be controlled by the actuation mechanism, such as a pull wire, rod, or other suitable actuating mechanism. A first, retracted configuration of legs 1010 may correspond to a first position of actuation members 1014, while a second, extended configuration of legs 1010 may correspond to a second position of actuation members 1014. To move legs 1010 from the first, retracted configuration to the second, extended configuration, the plurality of actuation members 1014 may be moved from the first position to the second position by the actuating mechanism. Any number of suitable legs 1010 may be used, and legs 1010 may be disposed approximately equidistant from each other, if desired. In one exemplary embodiment, four legs 1010 are utilized and may be formed by e.g., laser cutting distal tip 1012 at four equidistant longitudinal locations. The four longitudinal laser cuts may each continue along a radial direction that is substantially perpendicular to the longitudinal direction. Thus, each legs 1010 may have a generally curved longitudinal portion, and a pointed radial portion that is substantially perpendicular to the longitudinal portion. When in the first, retracted configuration, distal tip 1012 may be generally atraumatic.

Each leg 1010 may include an associated actuation member 1014 connected to each leg at, e.g., a distalmost end of distal tip 1012. It should be noted, however, that actuation member 1014 may connect to each leg 1010 at other suitable locations including, but not limited to the pointed radial portion of each leg 1010, or proximal to the distalmost end of each leg 1010. Legs 1010 may be simultaneously directed from the first, retracted configuration to the second, extended configuration. Alternatively, legs 1010 may be separately directed from the first, retracted configuration to the second, extended configuration. Distal tip 1012 may be biased closed, and pulled open with actuation members 1014 via, e.g., pull wires.

Legs 1010 may include features to engage and hold tissue, e.g., serrations or ridges, or other suitable features. It should be noted that legs 1010 may be utilized along any portion of a catheter to open or close a diameter to secure the catheter in place or to open a stricture. Legs 1010 may alternatively be utilized to grasp, pull, or move an object.

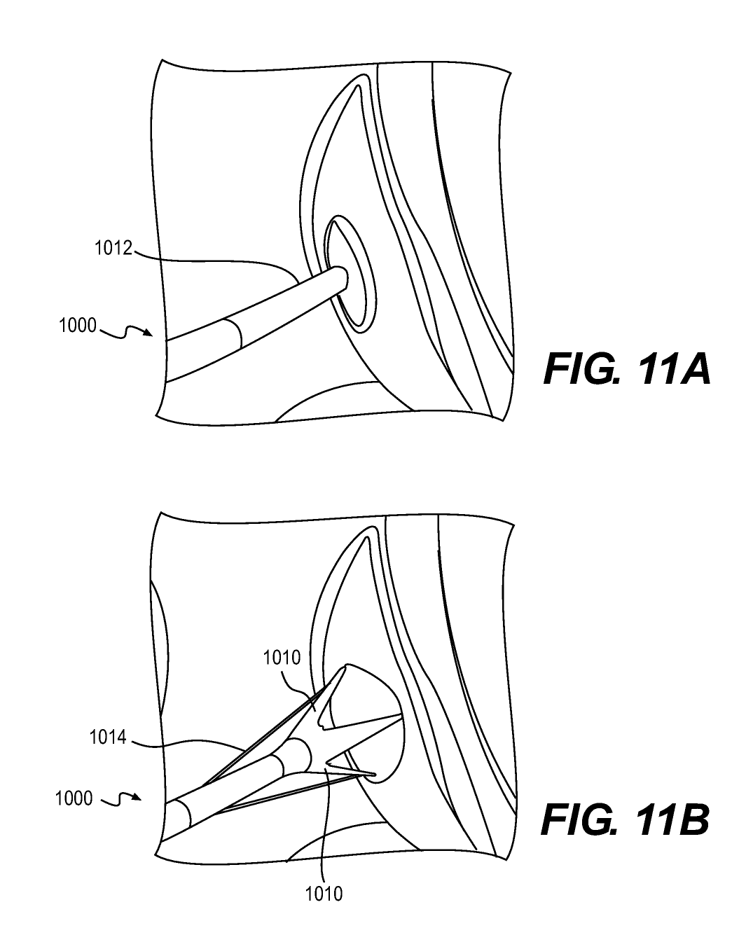

FIGS. 11A-11B illustrate various in vivo perspective views of medical device 1000. Specifically referring to FIG. 11A, medical device 1000 may be used with an endoscopic device described above with reference to FIGS. 3A and 3B. Medical device 1000 may extend through a lumen of the endoscopic device and out of the endoscopic device via an opening in a sidewall of the endoscopic device. Medical device 1000 may be directed out of the opening and into the papilla while legs 1010 are in the first, retracted configuration.

Referring to FIG. 11B, a user may direct the actuation members 1014 to the second position, causing legs 1010 to expand radially outward about the longitudinal axis of medical device 1000. As legs 1010 expand radially outward, an outer surface of legs 1010 may expand against an inner surface of the hepaticopancreatic ampulla, thus widening the papilla. A guidewire (not shown) may be passed through medical device 1000 to gain access to the common bile or biliary duct and/or the pancreatic duct.

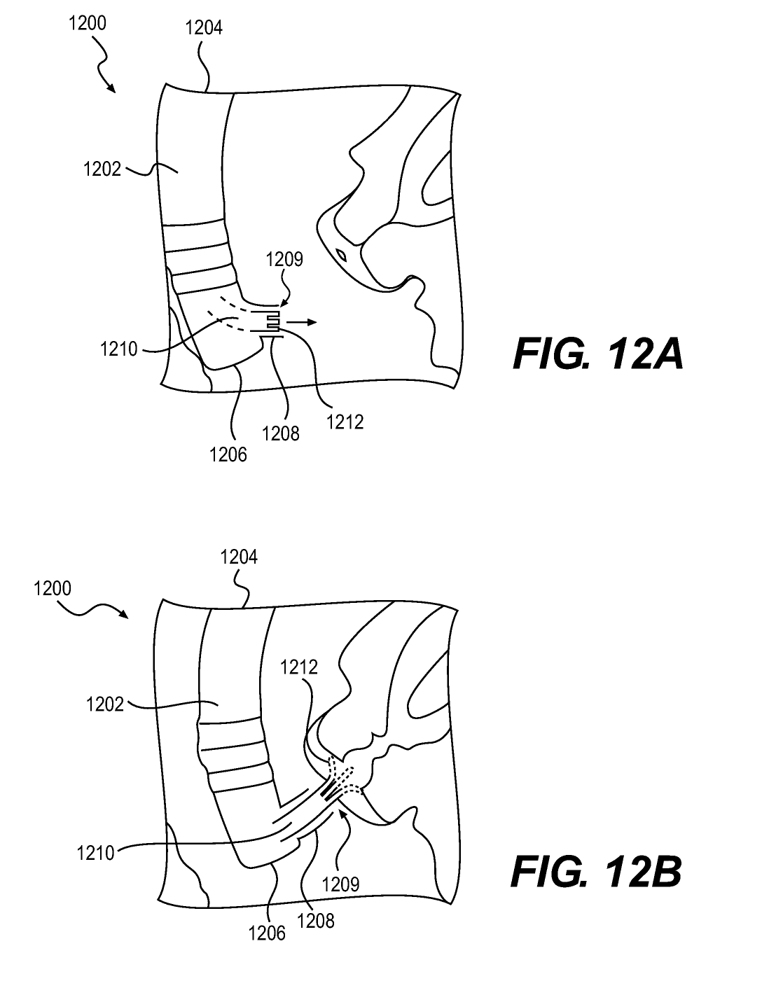

FIGS. 12A-12B illustrate various in vivo perspective views of an endoscopic device 1200. Endoscopic device 1200 may include an elongate member 1202 having a proximal end 1204, a distal end 1206, and a lumen (not shown) extending between proximal and distal ends 1204, 1206. Proximal end 1204 may be coupled to an appropriate handle (not shown), while distal end 1206 may include a tube 1208 defining an opening 1209 in communication with the lumen. In the embodiment shown, opening 1209 is shown as disposed in tube 1208. However, it should be noted that additional or alternative opening(s) 1209 may be disposed at other locations, such as, but not limited to a distal end face of endoscopic device 1200, a sidewall of endoscopic device 1200, or any other suitable location. In addition, tube 1208 may be a separate, distinct overtube or sheath slideable within endoscopic device 1200. A medical device 1210 may be disposed within the lumen of endoscopic device 1200 and through tube 1208. Medical device 1210 may include a plurality of legs 1212 at a distal end 1214 that, in a first configuration shown in FIG. 12A, is disposed within tube 1208. In the first configuration, legs 1212 may be biased toward a retracted position by, e.g., tube 1208 or a compression element (not shown). Medical device 1210 may be a hollow flexible tube having slots (formed by laser cutting, e.g.,) that define legs 1212. Medical device 1210 may include nitinol or other shape-memory materials that are pre-shaped so that legs 1212 may flare out when a biasing force acting on them is removed.

It should be noted that legs 1212 may alternatively be fins that are released and open when gaining access to the papilla. Alternatively, tube 1208 may include a catheter that has materials that can be expanded, similar to balloon dilation, to open the papilla and make it easier to gain access. In some embodiments, small guidewires could be passed through the tube 1208 to increase chances for passability. In some embodiments, tube 1208 may include a steerable distal section so that a user may have more confidence with the position of tube 1208. In some other embodiments, a wire may be removed so that a drilling device can be inserted to, e.g., drill through a stone to gain access. In such embodiments, an electrical connection may be provided.

Referring to FIG. 12B a user may direct the medical device 1210 out through opening 1209 via, e.g., an actuating handle (not shown) located at proximal end 1204 of endoscopic device 1200. As medical device 1210 protrudes out of opening 1209, the biasing force against legs 1212 may be removed, and legs 1212 may expand radially outward away from the longitudinal axis of medical device 1210. In the embodiment shown in FIG. 12B, opening 1209 may be aligned with the papilla such that when medical device 1210 exits opening 1209, legs 1212 expand within the hepaticopancreatic ampulla, widening the papilla.

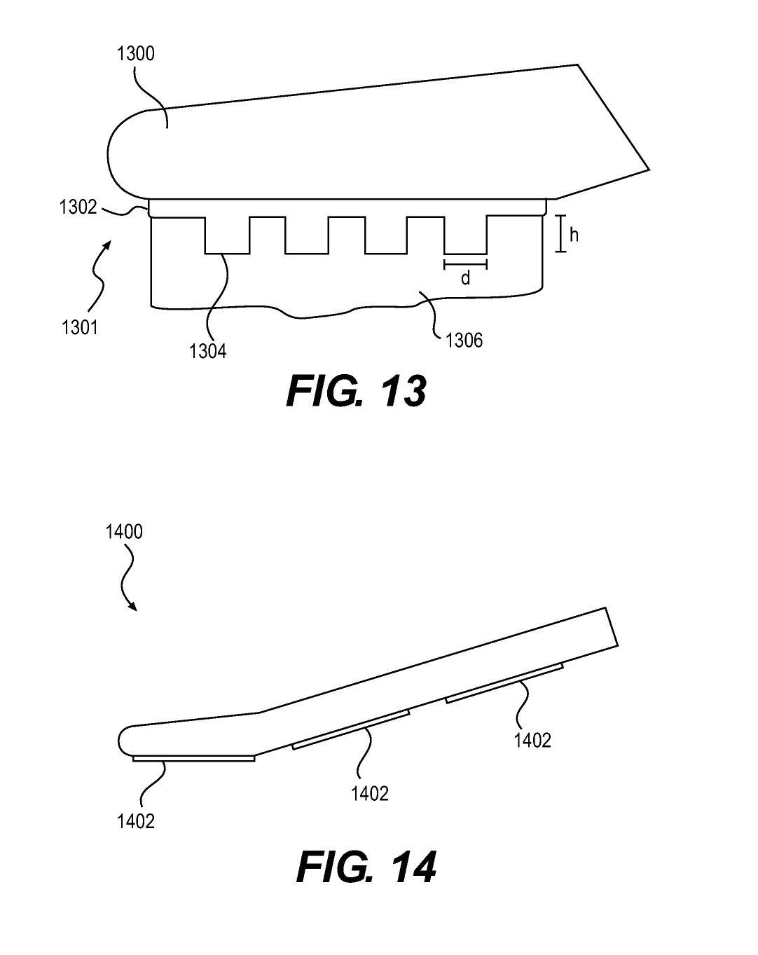

FIG. 13 is a partial side view of a patterned surface in accordance with an embodiment of the present disclosure. A medical device 1300 may include a coating 1301 having a base 1302 and plurality of protrusions (or micropillars) 1304. Each of coating 1301, base 1302, and protrusions 1304 may include one or more features of the similar components disclosed in U.S. Provisional Application No. 61/621,219, filed Apr. 6, 2012, and U.S. Nonprovisional application Ser. No. 13/857,998, filed Apr. 6, 2013, the entire disclosures of which are incorporated by reference herein. While depicted in FIG. 13 as having a cylindrical shape, protrusions having a rectangular or polygonal base, pyramids, or other non-traditional shapes are also contemplated.

In one embodiment, protrusions 1304 are cylinders that each have a diameter d and a height h measured from an outer surface of the base 1302 to a top surface of protrusions 1304. In at least one embodiment, the diameter d is between 1 μm and 100 μm. In one embodiment, the diameter d is between about 14 μm and 18 μm. In one embodiment, the diameter d of the protrusion is at least equal to its height h. In at least one embodiment, a ratio of height h of the protrusion 1304 to diameter d of the protrusion is between about 1 and 1.3. The protrusions may be spaced apart enough so that the tissue 1306 of a bodily vessel can fill the negative space between adjacent protrusions 1304. If the spacing is too small, the tissue may not be able to actually interlock. In at least one embodiment, the spacing between the protrusions may be dependent upon the particular type of tissue of the bodily vessel. In one embodiment, the spacings measured between the centers of one protrusion 1304 and an adjacent protrusion 1304 is greater than the diameter d of the one protrusion 1304. In at least one embodiment, the ratio of the spacings to the diameter d is between about 2.1 and 2.4.

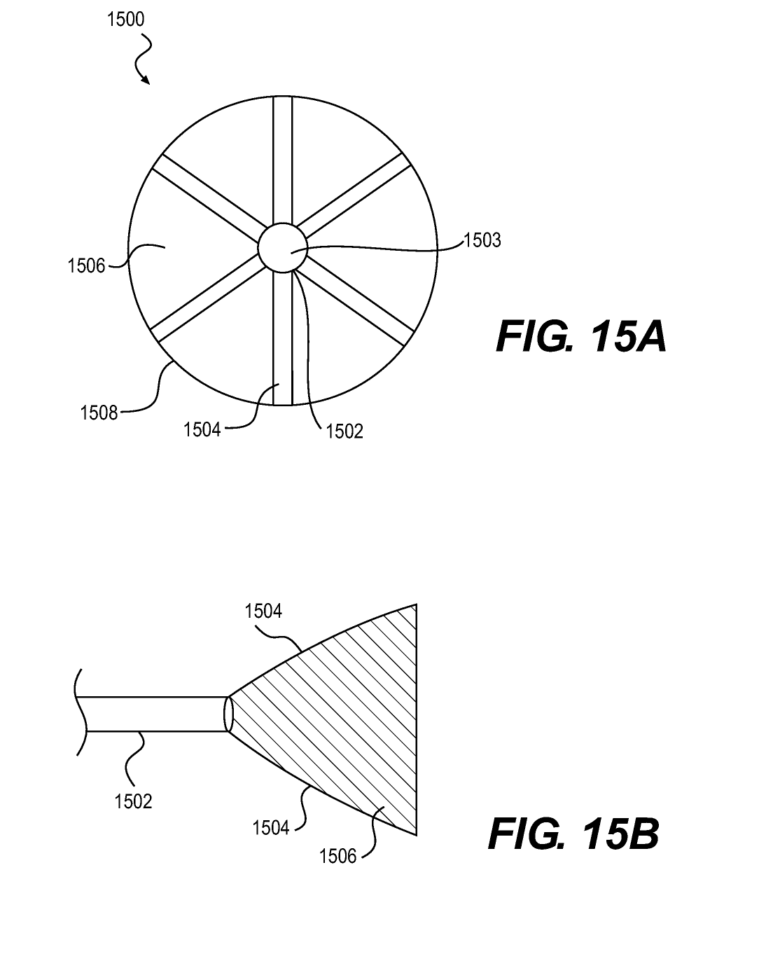

FIG. 14 illustrates an arrangement of the coating of FIG. 13 on a medical device in accordance with various embodiments of the present disclosure. FIG. 14 depicts an exemplary embodiment of a medical device 1400 having a plurality of coatings 1402 disposed along both a distal end and a length of medical device 1400. Each coating 1402 may be spaced apart from an adjacent coating 1402. However, it is contemplated that medical device 1400 may have one continuous coating extending from a distal end along its entire length, a single coating disposed at the distal end, or any other suitable arrangement.

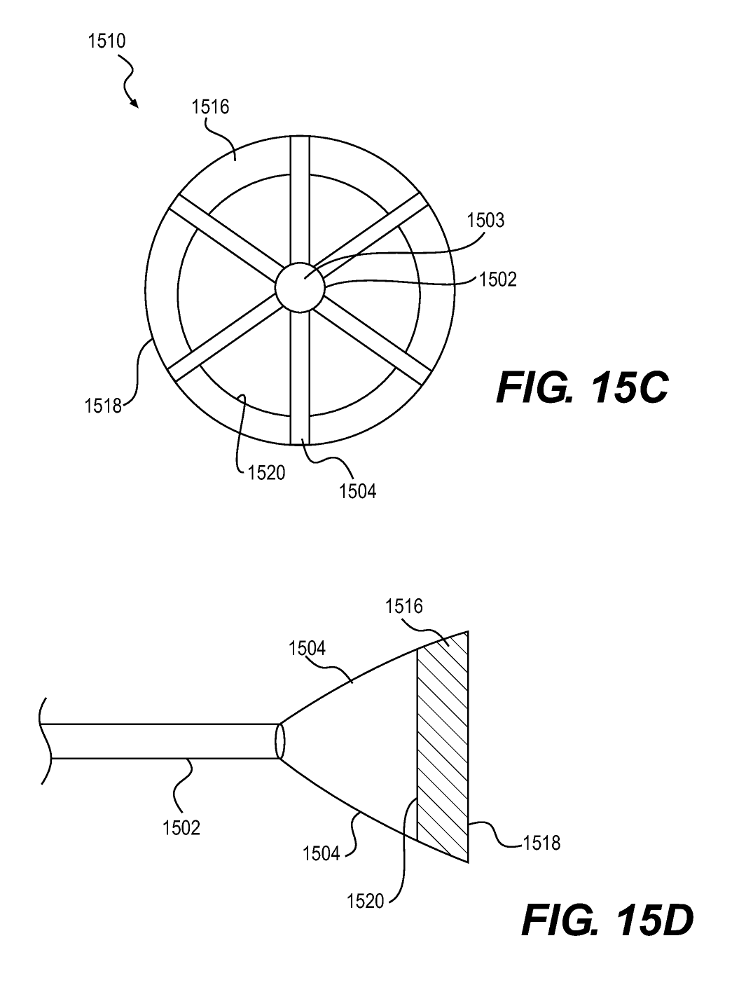

FIGS. 15A-15B depict a medical device 1500 according to an embodiment of the present disclosure. A plurality of legs 1504 may be coupled to a distal end of an elongate member 1502 having a lumen 1503. Any number of suitable legs 1504 may be used, and legs 1504 may be disposed approximately equidistant from each other, if desired. In one exemplary embodiment, six legs 1504 are utilized. Elongate member 1502 may be configured to allow medical devices, such as, e.g., a guidewire to pass through lumen 1503. A covering 1506 may be coupled to the distal ends of the plurality of legs 1504. Covering 1506 may be substantially similar to coating 1301 described above, and may extend from elongate member 1502 toward an outer surface 1508. As best seen in FIG. 15B, covering 1506 may resemble a suction cup. When the plurality of legs 1504 are in an expanded configuration, covering 1506 may also be in an expanded configuration to enhance contact with the bodily tissue, thereby increasing a stabilizing effect of medical device 1500. Medical device may be configured to accommodate a reciprocal movement of legs 1504 from the expanded configuration to a retracted configuration. When legs 1504 are in a retracted configuration, covering 1506 may similarly fold into a retracted configuration. Covering 1506 may enhance contact with bodily tissue to increase stabilization of the bodily tissue. The covering may be combined with other features that enhance the contact between medical device 1500 and the bodily tissue, including, but not limited to serrations, ridges, and the like. Covering 1506 may be optimized for specific types of bodily tissue.

FIGS. 15C-15D depict a medical device 1510 according to an embodiment of the present disclosure. Medical device 1510 may include an elongate member 1502, and a plurality of legs 1504 that are substantially similar to those described above in reference to medical device 1500. Any number of suitable legs 1504 may be used, and legs 1504 may be disposed approximately equidistant from each other, if desired. In one exemplary embodiment, six legs 1504 are utilized. Medical device 1510 may further include a covering 1516 coupled to the distal ends of the plurality of legs 1504. Covering 1516 may include similar features as covering 1506, but also have an outer surface 1518 and an inner surface 1520. Inner surface 1520 may have a diameter that is greater than a diameter of elongate member 1502 such that inner surface 1520 and elongate member 1502 do not directly contact each other. When the plurality of legs 1504 are in the expanded configuration, covering 1516 may also be in an expanded configuration to enhance contact with the bodily tissue, thereby increasing a stabilizing effect of medical device 1510. When legs 1504 are in the retracted configuration, covering 1516 may similarly fold into a retracted configuration. It is further contemplated that other suitable shapes and configurations may alternatively be utilized for distal end 1604.

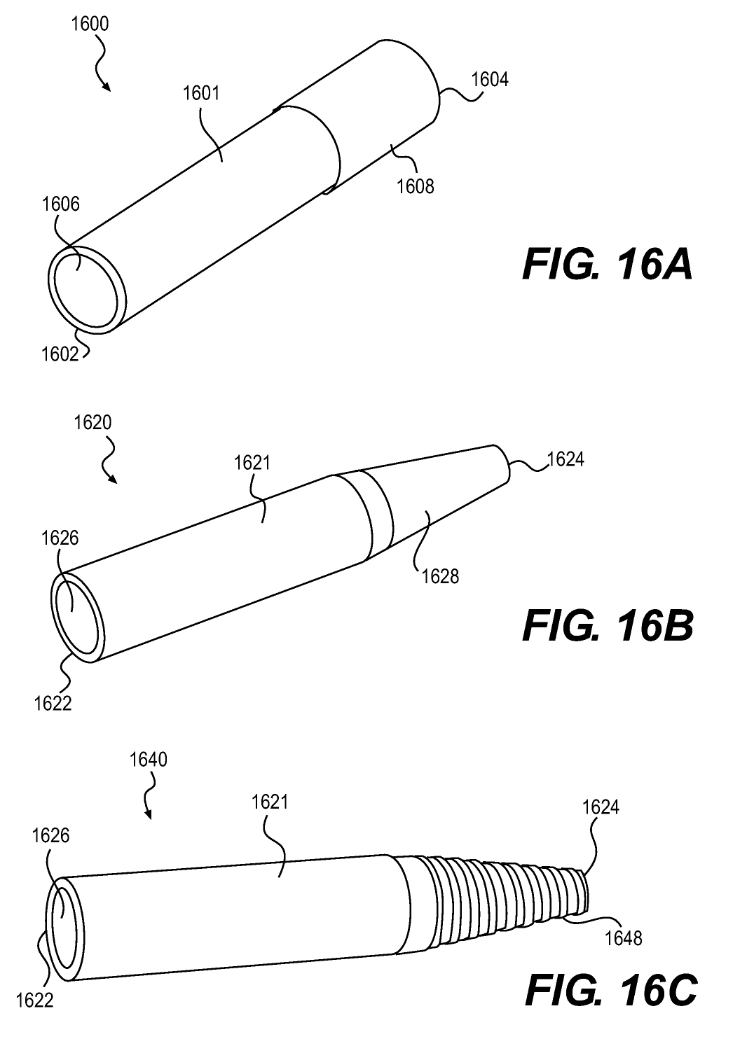

FIGS. 16A-16C illustrate exemplary embodiments of medical devices configured to improve access to the papilla. Referring to FIG. 16A, medical device 1600 may include a tip section of a cannula or sphincter tome. The tip section may include an elongate member 1601 with a proximal end 1602, a distal end 1604, and a lumen 1606 extending between the proximal and distal ends 1602, 1604. Medical device 1600 may further include a coating 1608 disposed around distal end 1604. Coating 1608 may include protrusions (or micropillars) and shapes substantially similar to those described with respect to coating 1301. In the embodiment of FIG. 16A, distal end 1604 may be cylindrical, and coating 1608 may be configured with a geometry that is tacky against bodily tissue. Because the geometry of coating 1608 is tacky against bodily tissue, once medical device 1600 is inserted through a narrow channel, such as, e.g., a hepaticopancreatic ampulla, when it is removed, the hepaticopancreatic ampulla may remain in a widened state, enabling a user to easily cannulate the common bile or biliary duct and/or the pancreatic duct further. It should be noted that coating 1608 may also be applied to an entire length or portion of medical device 1600.

Referring to FIG. 16B, medical device 1620 may include a tip section of a cannula or sphincter tome. The tip section may include an elongate member 1621 with a proximal end 1622, a distal end 1624, and a lumen 1626 extending between the proximal and distal ends 1622, 1624. Medical device 1620 may further include a coating 1628 disposed around distal end 1624 that is substantially similar to coating 1301. In the embodiment of FIG. 16B, distal end 1624 may be conical (or tapered) to facilitate entry into the papilla. Also, in the embodiment of FIG. 16B, coating 1628 may be configured with a geometry that is lubricous against bodily tissue. Because the geometry of coating 1628 is lubricous against bodily tissue, medical device 1620 may slide more easily into narrow openings, such as the papilla and the common bile or biliary duct and/or the pancreatic duct.

FIG. 16C illustrates a medical device 1640 that is substantially similar to medical device 1620, except that coating 1648 may be configured as a stranded coating wrapped in a spiral around distal end 1624. Because coating 1648 is in spiral form (e.g., in a helical pattern), medical device 1640 may be engaged or disengaged from a channel by rotational movement. It is further contemplated that other suitable shapes and configurations may alternatively be utilized for distal end 1624.

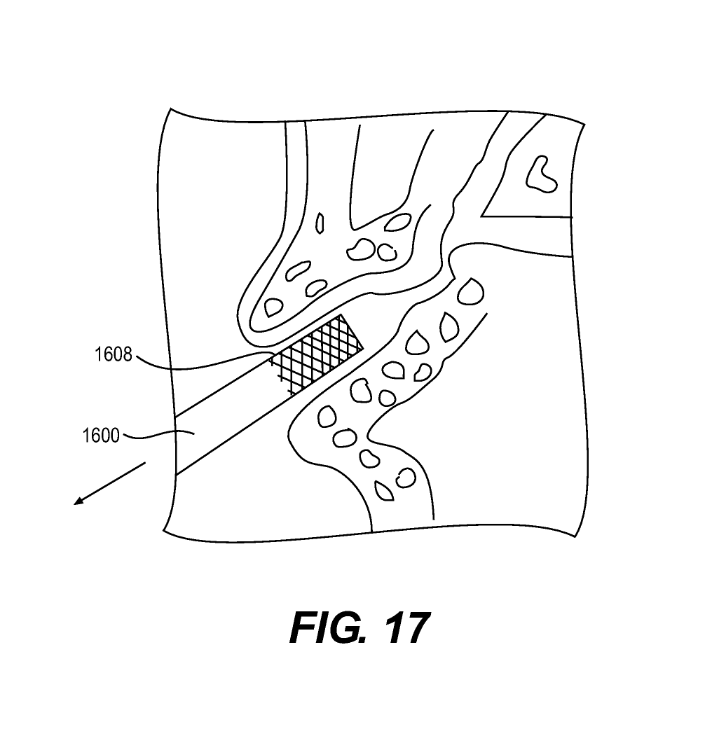

FIG. 17 illustrates an in vivo perspective view of medical device 1600 through a papilla and into either the common bile or biliary duct and/or the pancreatic duct. In the embodiment shown, coating 1608 is tacky against bodily tissue, and as medical device 1600 is removed from the hepaticopancreatic ampulla, the hepaticopancreatic ampulla may remain widened so that a user may easily further cannulate the common bile or biliary duct and/or the pancreatic duct.

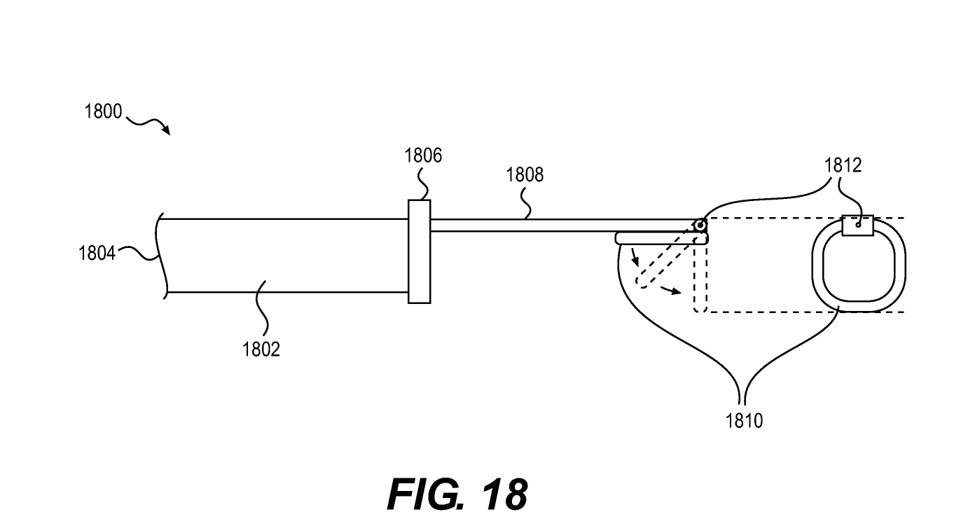

FIG. 18 shows a partial side view and an end view of a medical device for spreading tissue in accordance with an alternative embodiment of the present disclosure.

As shown in FIG. 18, a medical device 1800 may include an elongate shaft 1802 having a proximal end 1804 and a distal end 1806, and an actuating mechanism (not shown). A movable member 1808 may extend from distal end 1806, and a hoop 1810 may be disposed at a distal end of movable member 1808. Hoop 1810 may be coupled to the distal end of movable member 1808 via a joint 1812. Joint 1812 may be a pin or other suitable joint mechanism that allows for the reciprocal movement of hoop 1810 between a first, retracted position and a second, expanded position (dashed lines). Joint 1812 may be actuated by an actuating mechanism (not shown) located at proximal end 1804 of medical device 1800. In some embodiments, joint 1812 may be coupled to a ratcheting mechanism enabling hoop 1810 to move into the expanded position, and then back into the collapsed position when the ratcheting mechanism is released. In the first, retracted position, the plane of hoop 1810 may be substantially parallel to the longitudinal axis of medical device 1800, while in the second, expanded position, the plane of hoop 1810 may be substantially orthogonal to the longitudinal axis of medical device 1800. While in the second, expanded position, hoop 1810 may surround and stabilize an outer circumference of bodily tissue surrounding the papilla. For example, hoop 1810 may surround and isolate the papilla, preventing movement of the papilla by other bodily forces, and retract the surrounding tissue and open up the lumen for improved access. An inner circumference of hoop 1810 may include materials that assist in grasping the bodily tissue surrounding the papilla including, but not limited to roughened surfaces, hooks, and barbs. While the bodily tissue is stabilized, a user may access the papilla via a tool (not shown) disposed in a lumen of medical device 1800. Alternatively, the tool may be disposed within a lumen of an endoscopic device, or at another suitable location. A guidewire (not shown) may be passed through medical device 1800 to gain access to the hepaticopancreatic ampulla.

Any aspect set forth in any embodiment may be used with any other embodiment set forth herein. Every device and apparatus set forth herein may be used in any suitable medical procedure, may be advanced through any suitable body lumen and body cavity, and may be used to access tissue from any suitable body portion. For example, the apparatuses and methods described herein may be used through any natural body lumen or tract, including those accessed orally, vaginally, rectally, nasally, urethrally, or through incisions in any suitable tissue.

It will be apparent to those skilled in the art that various modifications and variations can be made in the disclosed systems and processes without departing from the scope of the disclosure. Other embodiments of the disclosure will be apparent to those skilled in the art from consideration of the specification and practice of the disclosure disclosed herein. It is intended that the specification and examples be considered as exemplary only. The following disclosure identifies some other exemplary embodiments.

We claim:

1. A medical device, comprising:
an elongate member having a proximal end and a distal end;
a plurality of legs coupled to the distal end of the elongate member; and
a moveable element distal to the distal end of the elongate member, disposed around the plurality of legs, and reciprocally moveable relative to the elongate member and the plurality of legs between a first position and a second position, wherein:
the plurality of legs are in a collapsed configuration when the moveable element is in the first position; and
the plurality of legs are in an expanded configuration when the moveable element is in the second position.

2. The medical device of claim 1, further comprising an actuating mechanism to move the movable element from the first position to the second position and, wherein the second position is proximal to the first position.

3. The medical device of claim 1, wherein the plurality of legs extend radially outward to move from the collapsed configuration to the expanded configuration.

4. The medical device of claim 1, wherein in the collapsed configuration, free distal ends of the plurality of legs are converged to penetrate a papilla.

5. The medical device of claim 1, wherein free distal ends of each of the plurality of legs includes a plurality of protrusions configured to engage bodily tissue atraumatically.

6. The medical device of claim 1, wherein the plurality of legs are substantially parallel to a longitudinal axis of the medical device while in the collapsed configuration.

7. The medical device of claim 6, wherein each of the plurality of legs includes a branch member at a distal end, wherein the branch member is angled toward the longitudinal axis.

8. The medical device of claim 7, wherein each of the plurality of legs has a wavy shape in the expanded configuration, and a pad coupled to the branch member.

9. The medical device of claim 1, wherein the plurality of legs form a cylindrical, distal tip in the collapsed configuration, wherein the moveable element is on a plurality of moveable elements, and at least one moveable element of the plurality of moveable elements is coupled to each of the plurality of legs.

10. The medical device of claim 1, further including a coating coupled to each of the plurality of legs, the coating including a plurality of protrusions.

11. The medical device of claim 10, wherein each of the plurality of protrusions has a diameter from about 1 μm to about 100 μm.

12. A method of using a medical device, comprising:
inserting an elongate member having a proximal end and a distal end into a body passage;
directing a plurality of legs coupled to the distal end of the elongate member toward a papilla while the plurality of legs are in a collapsed configuration; and
moving, in a proximal direction, a moveable element distal to the distal end of the elongate member from a first position to a second position to cause the plurality of legs to move from the collapsed configuration to an expanded configuration.

13. The method of claim 12, further comprising inserting a distalmost end of each of the plurality of legs into the papilla while the plurality of legs are in the collapsed configuration.

14. The method of claim 13, further comprising moving the moveable element from the first position to the second position while the distalmost end of each of the plurality of legs is within the papilla.

15. The method of claim 12, wherein the plurality of legs grasp and stabilize bodily tissue surrounding the papilla.

16. A method for expanding a papilla, comprising:
inserting a tube into a body passage, the tube having a proximal end, a distal end, and a lumen extending between the proximal and the distal ends;
directing an elongate member disposed within the lumen through the distal end of the tube and into the papilla; and
directing a plurality of legs disposed at a distal end of the elongate member between a collapsed configuration and an expanded configuration, wherein the plurality of legs:
are biased toward a longitudinal axis of the elongate member in the collapsed configuration; and
expand radially outward from the longitudinal axis in the expanded configuration.

17. The method of claim 16, further including inserting the tube and the elongate member through an endoscope.
